(19) United States
(12) Patent Application Publication
(10) Pub. No.: US 2019/0038514 A1
(43) Pub. Date: Feb. 7, 2019

(54) IN VIVO PLACING TUBE HAVING POSITIONING DEVICE

(71) Applicant: CHI MEI MEDICAL CENTER, TAINAN CITY (TW)

(72) Inventors: CHIEN-CHIN HSU, TAINAN CITY (TW); GWO-JIUN HORNG, TAINAN CITY (TW)

(21) Appl. No.: 16/051,799

(22) Filed: Aug. 1, 2018

(30) Foreign Application Priority Data
Aug. 4, 2017 (TW) 106126388

(51) Int. Cl. A61J 15/00; A61M 25/01

(52) U.S. Cl. CPC A61J 15/0003; A61M 25/0052; A61J 15/0073; A61M 25/0127

(57) ABSTRACT

An in vivo placing tube having a positioning device is disclosed herein. It comprises a tube having an invasive end and a handheld end communicated with the invasive end; a field coil buried in the invasive end of the tube; and an induced circuit board having an induction coil and a warning element electrically connected to the induction coil.

### BACKGROUND OF THE INVENTION

### 1. Field of the Invention

The present invention relates to an in vivo placing tube having a positioning device, the major function of which is to inform a medical staff of where the tube is currently positioned, so that the medical staff enables to clearly know the tubing placement progress for further increasing the speed and success rate of tubing insertion and placement in a target position in the body of a patient.

### 2. Description of Related Art

With the advancement of modern medicine, human beings increase their life expectancy through medical treatment. However, due to the uncontrolled eating habits and environmental changes of modern people, more diseases have emerged. For instance, dementia, severe gastroesophageal reflux, severe flatulence and the like, may cause patients to have eat and swallow difficulties. The patients who lose the ability to feed themselves will need to obtain necessary nutrients for their bodies through tube feeding.

Generally, the tubes placed in the human body are collectively referred to as “in vivo placing tubes”. For provision of human nutrition, the tube has one end placed in the stomach, so that the patient can directly absorb the nutrients delivered by the tube. When a nasogastric tube is used for feeding, it is inserted from the nasal cavity through the esophagus to the stomach, and the placement of the nasogastric tube is usually performed by a physician or a professional nurse instructed by a doctor.

The choice of the type of the tube placed in the body is mainly based on the disease of the patient. For instance, if the nasogastric tube is used, it needs to pass through a narrow space e.g. the nasopharynx and the esophagus, where it is greatly hindered while inserted.

At present, there are many ways to put the nasogastric tube into the human body. The most original placement method is to have the patient lie on his side or in a semi-recumbent posture and directly insert the nasogastric tube into the nasal cavity without any auxiliary tools. When the nasogastric tube reaches the stomach, it is necessary to determine whether the position of the tube is correct. For instance, an empty needle for feeding filled with 10-20 ml of air is used to inject air to the end of the nasogastric tube, and a stethoscope is used to place on the upper abdomen of the patient for auscultation. In case air sound incur to be heard, it means that the nasogastric tube is in the stomach. Tools can also be used to assist in the operation of the intubation. For instance, by placing a magnet at a front end of the nasogastric tube, when the nasogastric tube is inserted into the patient's body, the physician uses another magnet to guide the nasogastric tube outside the body. For instance, a China Patent No. CN105920721A, issued on 7 Sep. 2016, has disclosed a nasointestinal tube. It comprises a parenteral catheter for insertion and uses a magnet and a guiding wire to drive the nasointestinal tube to move so as to speed up the intubation operation. However, the above mentioned methods do not reflect a high success rate.

Furthermore, the endoscope can be used in combination with the in vivo placing tube. The endoscope is placed from the oral cavity to reach the stomach together with the nasogastric tube that enters through the nasal cavity. The image transmitted by the endoscope allows the physician to know the current position of the nasogastric tube more clearly. Although these endoscope-based intubation methods can effectively improve the success rate, they are necessary to insert an additional endoscope into the patient's mouth, which may cause discomfort to the patient. The use of radiation may make the patient feel uneasy about exposure to the radiation environment. Therefore, there is an urgent need to develop an in vivo placing tube that is easy to intubate and has a high success rate to avoid additional discomfort to the patient.

### SUMMARY OF THE INVENTION

In view of the above-mentioned problems, the object of the present invention is to provide an in vivo placing tube having a positioning device which can immediately inform a medical staff of where the tube is currently positioned so as to help the medical staff to clearly know a tubing placement progress and avoid the tubing from placement in an improper position in the body of a patient, such as the trachea, causing complications.

Disclosed herein is an in vivo placing tube having a positioning device. It mainly comprises a tube having an invasive end and a handheld end communicated with the invasive end; a field coil buried in the invasive end of the tube; and an induced circuit board having an induction coil for generating a voltage by electromagnetic induction of the field coil and a warning element electrically connected to the induction coil.

According to an embodiment of the present invention, the tube is made of silicone or polyethylene (PE).

According to an embodiment of the present invention, a power supply module is further provided and electrically connected to the field coil by an electric wire embedded in the tube.

According to an embodiment of the present invention, the warning element is a buzzer or an LED.

According to an embodiment of the present invention, the induced circuit board further comprises a statistical wafer.

According to an embodiment of the present invention, the field coil has a frequency ranging from 120 kHz to 140 kHz.

According to an embodiment of the present invention, the in vivo placing tube having a positioning device is a nasogastric tube, a nasointestinal tube, a urinary tube or a chest tube.

According to an embodiment of the present invention, a power supply module is further provided at the handheld end and electrically connected to the field coil.

### BRIEF DESCRIPTION OF THE DRAWINGS

FIG. 1 is a stereogram showing an in vivo placing tube having a positioning device according to the present invention;

FIG. 2 is a schematic diagram showing an in vivo placing tube having a positioning device in use according to the present invention.

### DETAILED DESCRIPTION OF THE PREFERRED EMBODIMENT

Hereinafter, an exemplary embodiment of the present invention will be described in detail with reference to the accompanying drawings.

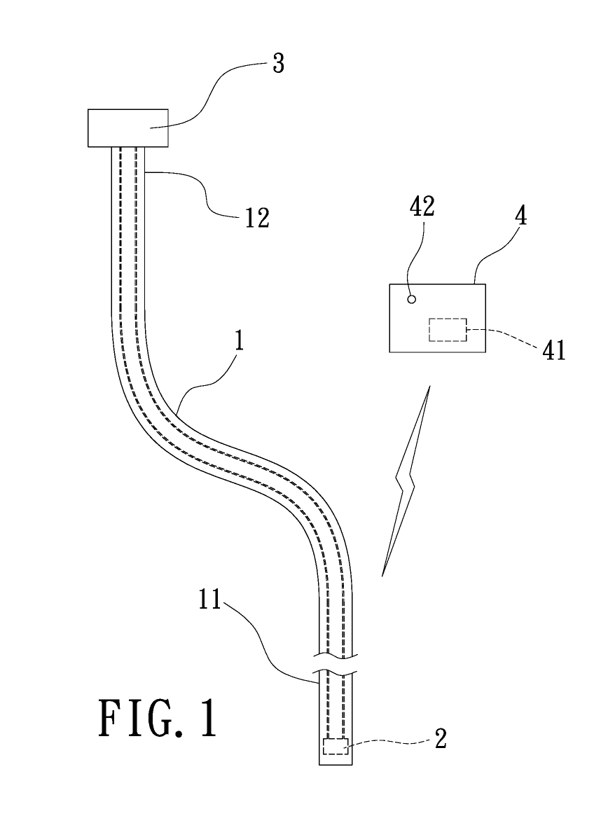

As showed in FIG. 1, a stereogram showing an in vivo placing tube having a positioning device according to the present invention is disclosed herein. It mainly comprises a tube (1) having an invasive end (11) and a handheld end (12) communicated with the invasive end (11); a field coil (2) buried in the invasive end (11) of the tube (1), wherein the tube (1) is made of silicone or polyethylene (PE); and an induced circuit board (4) having an induction coil (41), a warning element (42) electrically connected to the induction coil (41) and a statistical wafer.

Accordingly, by electromagnetic induction, the induction coil (41) of the induced circuit board (4) generates an induced electromotive force to actuate the warning element (42) and thus achieves the effect of reminding. Preferably, the warning element (42) is a buzzer or an LED to attract the attention of the physician. The field coil (2) has a frequency ranging from 120 kHz to 140 kHz. If the magnetic field strength is insufficient to generate electromagnetic induction, a power supply module (3) is further disposed at the handheld end (12) of the tube (1) and electrically connected to the field coil (2) by an electric wire embedded in the tube (1). The power supply module (3) provides electric power to the field coil (2) to enhance the induced electromotive force.

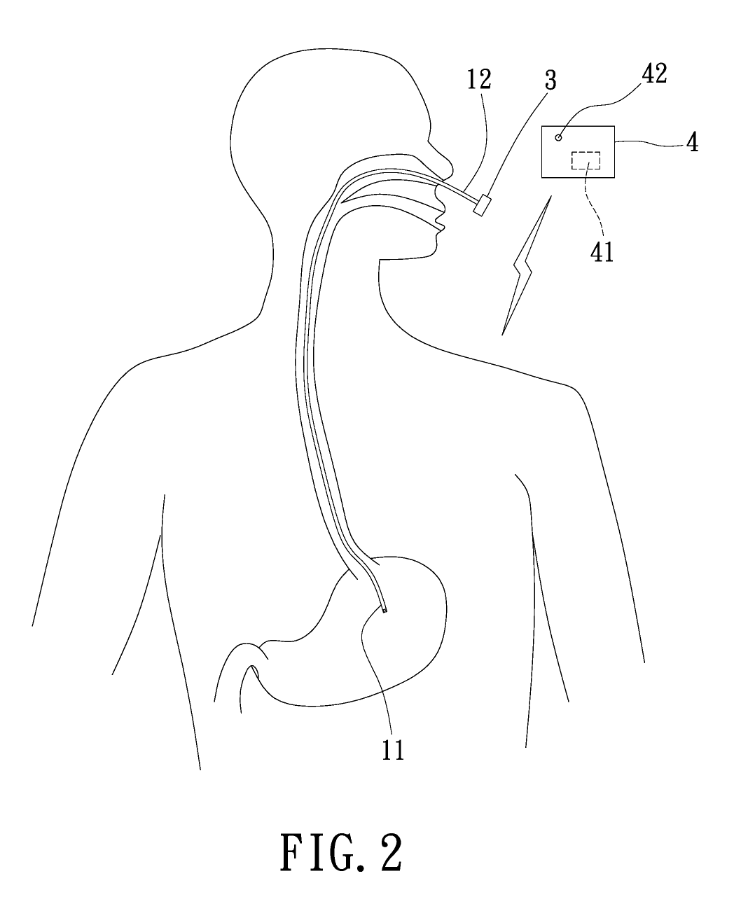

Referring to FIG. 1 and FIG. 2, the in vivo placing tube is a nasogastric tube as an example. In practical use, the handheld end (12) of the tube (1) is held by the physician, and the invasive end (11) is inserted from the nasal cavity of the patient, through the esophagus to the stomach. During the intubation process, the field coil (2) buried in the invasive end (11) generates a magnetic field, and the induced circuit board (4) held by the physician is close to the patient's body. The distance between the induced circuit board (4) and the patient's body is about 10-15 cm. When the induction coil (41) is close to the field coil (2), the field coil (2) generates the frequency ranging from 120 kHz to 140 kHz, which causes the induction coil (41) to generate a voltage due to the principle of electromagnetic induction for further actuating the warning element (42) electrically connected to the induction coil (41). If the frequency of the field coil (2) is higher than 140 kH or lower than 120 kHz, the induction coil (41) cannot generate a voltage. Generally, the LED can be used as the warning element (42). When the LED is actuated to illuminate, the physician enables to know where the invasive end (11) of the nasogastric tube is positioned in the patient.

If the magnetic field strength is insufficient for the induction coil (41) of the induced circuit board (4) to generate electromagnetic induction, the power supply module (3) at the handheld end (12) of the tube (1) is turned on so as to supply additional electric power to the field coil (2) via the electric wire buried inside the tube (1) and thus achieve the purposes for smoothly generating electromagnetic induction and enhancing the induced electromotive force.

In such a case, the physician can quickly adjust the position of the nasogastric tube according to the LED light, and accurately makes the invasive end (11) of the tube (1) reach the area of the stomach. After the tubing placement progress of the nasogastric tube is completed, the patient can be fed through the nasogastric tube over the next few weeks to supplement the necessary nutrients for his body.

Additionally, the statistical wafer of the induced circuit board (4) has a function of recording the nasogastric tube used by the patient. One patient may have replaced more than one nasogastric tube due to his or her own physical condition, and the suitable service life of each nasogastric tube varies according to the patient's physical condition. Therefore, the statistical wafer of the induced circuit board (4) is activated in advance before the tube (1) is placed in the patient. In such a case, the information is first sent back to the physician's or the nurse's electronic device, allowing the medical staff to learn how long the nasogastric tube that the patient is using has been used in the past. The statistical wafer of the induced circuit board (4) can also count how many nasogastric tubes have been used by the patient so that the medical staff can better understand the condition of the patient.

Compared with the technique available now, the present invention is has the following advantages:

1. The present invention having the field coil and the induction coil utilizes the phenomenon of electromagnetic induction, so that the electrified field coil causes the induction coil to generate an induced electromotive force to actuate the warning element electrically connected to the induced circuit board, which is an innovative technique to connecting the inside and outside of the human body by induction.

2. The induced circuit board of the present invention can be used as a positioning device, and its induction coil that has generated electric power can make the warning component act different reminding effects, e.g. sound, lighting and the like, so that the medical staff enables to clearly know where the tube is currently positioned in the body based on the effect produced for further increasing the speed and success rate of tubing insertion and placement in the target position in the body of a patient. Furthermore, different from prior art which requires using an additional endoscope, radiation and the like, to visually recognize the position of tubing insertion and placement in the body, the present invention does not require additional insertion of other objects into the patient, so the present invention can avoid additional discomfort to the patient.

What is claimed is:

1. An in vivo placing tube having a positioning device, comprising:
a tube having an invasive end and a handheld end communicated with the invasive end;
a field coil buried in the invasive end of the tube, wherein the field coil has a frequency ranging from 120 kHz to 140 kHz; and
an induced circuit board having an induction coil for generating a voltage by electromagnetic induction of the field coil and a warning element electrically connected to the induction coil.

2. The in vivo placing tube having a positioning device as claimed in claim 1, wherein the tube is made of silicone or polyethylene (PE).

3. The in vivo placing tube having a positioning device as claimed in claim 1, further comprises a power supply module electrically connected to the field coil by an electric wire embedded in the tube.

4. The in vivo placing tube having a positioning device as claimed in claim 1, wherein the warning element is a buzzer or an LED.

5. The in vivo placing tube having a positioning device as claimed in claim 1, wherein the induced circuit board further comprises a statistical wafer.

6. The in vivo placing tube having a positioning device as claimed in claim 1, is a nasogastric tube, a nasointestinal tube, a urinary tube or a chest tube.

7. The in vivo placing tube having a positioning device as claimed in claim 1, further comprises a power supply module disposed at the handheld end and electrically connected to the field coil.
